(19) United States
(12) Patent Application Publication
(10) Pub. No.: US 2021/0304846 A1
(43) Pub. Date: Sep. 30, 2021

(54) Personalized compounding of therapeutic components and tracking of their influence on a measured parameter using a complex interaction model

(71) Applicant: Stephen J. Brown, Malibu, CA (US)

(72) Inventor: Stephen J. Brown, Malibu, CA (US)

(21) Appl. No.: 16/926,350

(22) Filed: Jul. 10, 2020

Related U.S. Application Data
(60) Provisional application No. 63/001,422, filed on Mar. 29, 2020.

(51) Int. Cl. G16B 40/00; G06K 9/62; G06N 5/02

(52) U.S. Cl. CPC G16B 40/00; G06K 2209/05; G06N 5/02; G06K 9/6276

(57) ABSTRACT

A system and a method for a computer-implemented adaptive machine learning approach for tracking the effects of a therapeutic compound formulation containing a number of therapeutic components on one or more measurable parameters of a patient. The therapeutic components are represented by vectors in a complex interaction space. The vectors are not dimensionally reduced during the tracking process and they are adapted for tracking correlations between the therapeutic components, as those manifest in the values of the one or more measurable parameters. The approach is useful for making personalized adjustments of the therapeutic components of the compound formulation based on the one or more measurable patient responses captured by the measurable parameters.

### FIELD OF THE INVENTION

The present invention relates generally to methods and systems that deploy adaptive machine learning algorithms and a complex interaction model to track a measured parameter indicative of a patient's response to a personalized compound of therapeutic components.

### BACKGROUND OF THE INVENTION

There is a considerable body of prior art addressing machine learning approaches to customize treatment of patients. Such algorithms may process vast amounts of data including historical records to determine optimal treatment approaches. Many of these approaches attempt to build a mechanistic model or a physics-based model (e.g., using models from statistical physics) to follow the activity and interactions of the therapeutic components administered to the patient.

Another group of learning approaches adopt an agnostic approach to the underlying mechanisms. A common issue with such approaches is their convergence on a quasi-causal interaction model that is classical in nature but bears little insight on the dynamics of the compound activity. Furthermore, such models can fall into an annealed minimum and lose valuable information (e.g., through dimensional reduction and causal network inference).

What is needed is a model that tracks the performance of a personalized compound formulation consisting of therapeutic components without imposing unnecessary limitations that incur the disadvantages associated with prior-art systems.

### SUMMARY

The present invention addresses the shortcomings of the prior art by presenting a method and a computer-implemented adaptive learning approach in which the therapeutic components of a therapeutic compound formulation administered to a patient have a richer representation in a complex interaction space. This representation is well-suited for tracking correlations between the therapeutic components and it does not apply dimensional reduction. Further, the approach of the invention is useful for making personalized adjustments of the therapeutic components of the compound formulation based on one or more measurable patient responses.

The method of invention is designed for personalized adjustment of the chosen therapeutic components of the therapeutic compound formulation based on a measurable patient response, or a macro-parameter for which the patient can be measured. A non-exhaustive list of such measurable patient responses includes cytokines, respiratory functions, inflammation conditions, vital signs and other macro-parameters that can be obtained through measurement. The therapeutic components can include, among other, corticosteroids, antivirals, antioxidants and immunoglobulins. Preferably, the therapeutic components are chosen in an initial formulation procedure based on information or data obtained from a clinical data repository. The data can include patient profile, infection pathology, therapeutic availability, counterindications, genotype and other information specific to the patient or patient type.

The method of invention assigns the measurable patient response that is selected for the given patient to a one-dimensional response measure space. When relying on more than one measurable patient response, then each of them is assigned to its own one-dimensional response measure space. A suitable one-dimensional space is a real space.

When administering a first therapeutic component to the patient, it is important to know or to estimate a first expected initial effect on the measurable patient response. This may be done by relying on information from the clinical data repository for the specific patients, the type or group into which the patient belongs, or even from information collected directly from the patient.

In accordance with the method, the first therapeutic component is represented as a first vector in the complex interaction space. The first vector has a magnitude proportional to an initial amount of the first therapeutic component administered to the patient. The first vector also has a projection onto the one-dimensional response measure space. The projection is set to be equal to the first expected initial effect. Although the interaction space can be literally complex, i.e., it may deploy real and imaginary dimensions, alternative mathematical equivalents not expressly using imaginary numbers (where the imaginary number i=\sqrt{−1}) can be used as well.

As the measurable patient responses are collected with appropriate measurement devices or monitors, the method calls for making adjustments to a complex phase of the first vector to match the patient responses. In general, more than one complex phase adjustment will match the patient response. However, all possible complex phase adjustments to the first vector are kept rather than being discarded or “collapsed”. This step is important in order to prevent a dimensional reduction in the representation that would reduce the ability to track correlations.

In particular, tracking correlations is important when administering a second therapeutic component and still additional therapeutic components that can make up the therapeutic compound formulation. The second therapeutic component has a second expected initial effect on the measurable patient response based on the information from the clinical data repository. This second therapeutic component is represented as a second vector in the complex interaction space. Again, the magnitude of the second vector is proportional to an initial amount of the second therapeutic component while its projection onto the one-dimensional response measure space is equal to the second expected initial effect.

The adjustments in complex phases of the first and second vectors are determined so as to match the measurable patient response. Furthermore, the correlations between the first and second therapeutic components are tracked in the complex interaction space without applying dimensional reduction, i.e., by tracking all the possible relationships of the first and second vectors and more specifically of their complex phases in the interaction space that yield the measured patient response.

Preferably, the determination of adjustments in the complex phases of the vectors is performed with an adaptive learning algorithm. The learning algorithm is conditioned to retain the full vector representations without dimensional reduction. Also, the adaptive learning algorithm is conditioned not to set or learn causal connections, but to instead continue to work with the full representation that posits only correlations. The adaptive learning algorithm can produce a history of the measured patient responses and base the adjustments in complex phases of the first, second and any additional vectors representing still other additional therapeutic components on the history. Preferably, the total number of additional therapeutic components is relatively small in order to prevent an explosion in the total correlations that have to be tracked. For example, the total number of these additional therapeutic components is kept at less than 20. Additionally, the measurable patient response should be monitored or measured at a higher than normal rate or frequency in order to provide sufficient data to the adaptive learning algorithm.

In practice, the therapeutic compound formulation is prepared by an immunomodulator therapeutic compounding module. Also, the adaptive learning method is preferably implemented on a computer designed to make personalized adjustments in the therapeutic components that constitute the therapeutic compound formulation. The computer-implemented adaptive learning method calls for assigning the one-dimensional response measure space to the selected measurable patient response and setting the first expected initial effect from the data obtained from the clinical data repository. The learning of the adjustments to the complex phases of first, second and any additional vectors takes place in the complex interaction space. The correlations found in that space are kept without presuming or setting of causal connections and without dimensional reduction being applied. This avoids annealing of the adaptive learning algorithm to a potential local minimum. In other words, falling into a minimum of an optimization function typically experienced by a typical machine learning algorithm is avoided.

The present invention, including the preferred embodiment, will now be described in detail in the below detailed description with reference to the attached drawing figures.

### BRIEF DESCRIPTION OF THE DRAWING FIGURES

FIG. 1 is a schematic diagram illustrating a system for administering a therapeutic compound formulation to a patient in accordance with the invention

FIG. 2 is a diagram illustrating a representation of therapeutic components and a response measure space in a complex interaction space

FIG. 3 is a diagram illustrating a method for adjusting a therapeutic compound formulation in accordance with the invention

FIG. 4 is a schematic diagram of a compounding controller for preparing initial and adjusted therapeutic compound formulations

FIG. 5 is a schematic diagram of an inflammation response monitor for measuring patient responses

FIG. 6 is a diagram illustrating an overall approach to adaptive control and adaptive machine learning in accordance with the invention

FIG. 7 is a diagram illustrating another exemplary representation of therapeutic components and a response measure space in a higher-dimensional complex interaction space

### DETAILED DESCRIPTION

The figures and the following description relate to preferred embodiments of the present invention by way of illustration only. It should be noted that from the following discussion, alternative embodiments of the structures and methods disclosed herein will be readily recognized as viable alternatives that may be employed without departing from the principles of the claimed invention.

Reference will now be made in detail to several embodiments of the present invention, examples of which are illustrated in the accompanying figures. It is noted that wherever practicable, similar or like reference numbers may be used in the figures and may indicate similar or like functionality. The figures depict embodiments of the present invention for purposes of illustration only. One skilled in the art will readily recognize from the following description that alternative embodiments of the structures and methods illustrated herein may be employed without departing from the principles of the invention described herein.

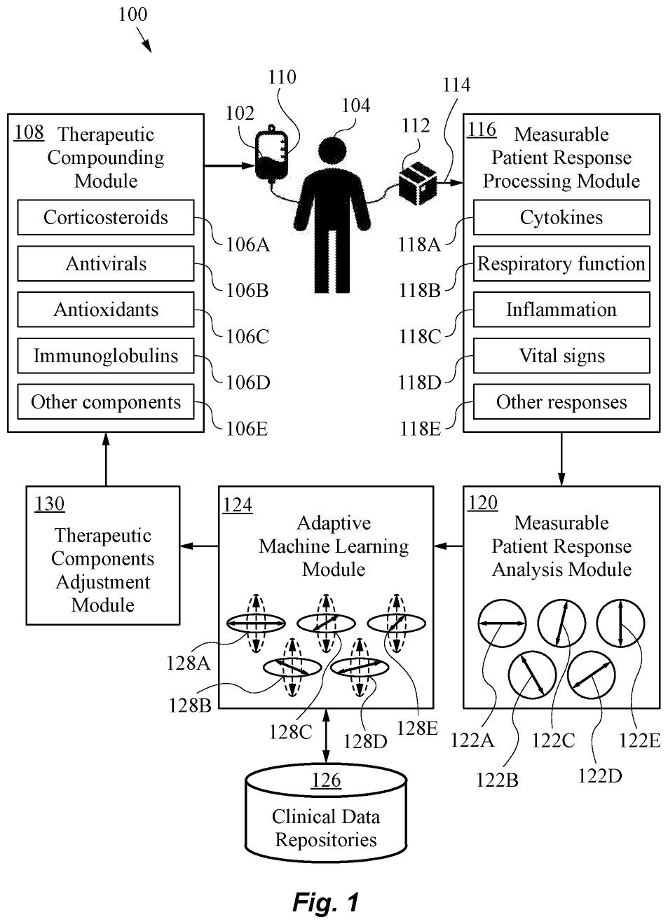

FIG. 1 shows an exemplary system 100 for practicing a method of administering a therapeutic compound formulation 102 to a patient 104 and adjusting therapeutic components 106A-E combined or compounded in formulation 102 in accordance with an embodiment of the invention. A therapeutic compounding module 108 performs the actual step of compounding formulation 102 and providing it for administration to patient 104 with a suitable administration mechanism 110, e.g., an intra-venous administration mechanism in the present example.

Although specific therapeutic components 106A-E are shown in FIG. 1, in general therapeutic components 106 will vary depending on the condition, affliction or disease of patient 104. The method of invention is most useful in situations where patient 104 requires administration of a combination, a cocktail or an otherwise mixed, amalgamated, combined or compounded collection of therapeutic components 106. More particularly, the method is advantageous when therapeutic compound formulation 102 is to be varied or adjusted in a personalized manner.

System 100 has measurement devices or monitors 112 for collecting, measuring or monitoring one or more measurable patient responses 114 which are macro-parameters that are ascertained by measures that are based on statistically significant events or conditions, e.g., body temperature, red blood count, antibody count, heart rate etc. Devices 112 are chosen in accordance with measurable patient responses 114 that are used in the method. The output of devices 112 is supplied to a measurable patient response processing module 116. Processing module 116 has a number of specific measurement processing units 118A-E for conditioning and analyzing corresponding measured patient responses 114.

In the present example the chosen measurable patient responses 114 are macro-parameters in the sense that they are supported by statistically significant events/conditions. These measurable patient responses 114 include a level of cytokines, a respiratory function, a level of inflammation, vital signs (e.g., pulse rate, blood pressure, etc.) and other selected patient responses not specifically enumerated. All measurable patient responses 114 are delivered to processing module 116 to distribute to measurement processing unit 118A for analyzing and processing the level of cytokines, to unit 118B for analyzing and processing the respiratory function, to unit 118C for analyzing and processing the level of inflammation and to unit 118D for analyzing and processing the vital signs (e.g., pulse rate, blood pressure, etc.). Measurement processing unit 118E is supplied by processing module 116 with still other measurable patient responses 114 monitored by measurement devices 112. Many additional and/or different measurement processing units 118 may be deployed by patient response processing module 116 depending on what patient responses 114 are subject to measurement and/or monitoring.

Measurable patient response processing module 116 is connected to measurable patient response analysis module 120. Analysis module 120 adopts a representation of measurable patient responses 114 that maps or assigns each one of them to its own one-dimensional response measure space 122A-E. Preferably, response measure spaces 122A-E are one-dimensional real spaces. Further, spaces 122A-E are separate or disjoint such that no projection or overlap is found between any of them.

Analysis module 120 is further connected to an adaptive machine learning module 124. Machine learning module 124 adopts in its learning approach or algorithm the irreducible representation of response measure spaces 122A-E set in analysis module 120. Also, machine learning module 124 is connected to clinical data repositories 126 that supply important information or data about patient 104. Specifically, important data include patient profile, infection pathology, therapeutic availability, counterindications, genotype and other data that is either specific to patient 104 or a patient type or group to which patient 104 may belong. Clinical data repositories 126 can be embodied by a single local database or a number of non-collocated databases on various servers and other infrastructure.

Machine learning module 124 uses data from clinical data repository 126 to select an initial formulation procedure that dictates the initial amounts of therapeutic components 106A-E to be compounded into therapeutic compound formulation 102. Specifically, when administering first, second and additional therapeutic components 106A-E to patient 104, it is important to know or at least to estimate a first expected initial effect on each measurable patient response 114 that will be affected by administered therapeutic components 106A-E. While this may be done by relying on patient-specific information from clinical data repositories 126 for some patients, for other patients the available information may only include patient type or group to which they belong. In still other cases, if neither type of information is available in clinical data repositories 126, or if the available data is incomplete or otherwise insufficient, the relevant data may be collected directly from patient 104 and entered into clinical data repositories 126.

In accordance with the method of invention, adaptive machine learning approach implemented by learning module 124 places or represents therapeutic components 106A-E of compound formulation 102 in a complex interaction space 128. In the present example, complex interaction space 128 is actually broken up into five separate complex interaction spaces 128A-E, these may be considered subspaces, such that each subspace is assigned to one response measure space 122A-E, respectively. This approach allows for a richer representation of therapeutic components 106A-E because of the at least one additional dimension afforded by imaginary axis Im indicated by a dashed arrow in each one of the complex interaction spaces 128A-E or subspaces.

It should be noted that this at least one additional dimension does not have to be expressed with imaginary numbers. Although interaction spaces 128A-E can be literally complex, i.e., they may deploy real and imaginary dimensions, alternative mathematical equivalents not expressly using imaginary numbers (where the imaginary number i=\sqrt{−1}) can be used as well. However, the additional dimension postulated in interaction spaces allows for a richer representation of vectors associated with therapeutic components 106A-E and is well-suited for tracking correlations between therapeutic components 106A-E.

System 100 has a therapeutic components adjustment module 130 connected to adaptive machine learning module 124 and to therapeutic compounding module 108. Adjustment module 130 is configured to apply adjustments in to therapeutic components 106A-E based on the outputs of machine learning module 124.

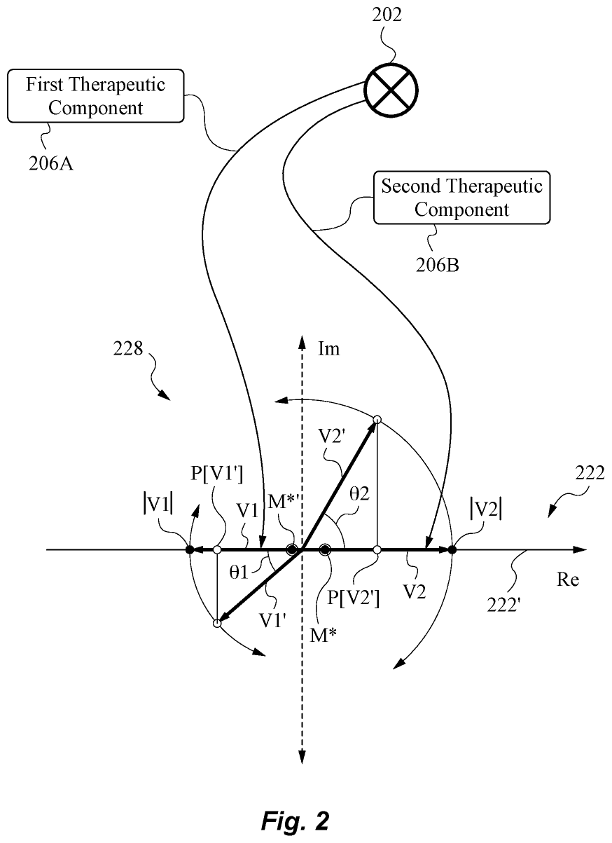

FIG. 2 is a diagram illustrating a general case for how therapeutic components of compound formulations are represented according to the method of invention. In this general case a first therapeutic component 206A and a second therapeutic component 206B belonging to a compound formulation 202 are represented in a complex interaction space 228. In a particular case, therapeutic components 206A and 206B may be therapeutic components 106A, 106B of compound formulation 102 from the embodiment of FIG. 1.

In FIG. 2 a particular measurable patient response 214 is mapped to or represented in a response measure space 222 that is assigned to a real and one-dimensional space spanned by axis 222′. The origin of axis 222′ is placed at the value of measurable patient response 214 prior to the administration of therapeutic components 206A and 206B.

Upon administration, first therapeutic component 206A is represented by a first vector V1 in complex interaction space 228. The magnitude of first vector V1 is set in proportion to an initial amount of first therapeutic component 206A administered to the patient. In the simplest case, as shown in FIG. 2, the magnitude of first vector V1 is simply set equal to first expected initial effect of first therapeutic component 206A on measurable patient response 214. Further, first vector V1 also has a projection onto one-dimensional response measure space 222, in this case a projection onto axis 222′, that is set equal to first expected initial effect of first therapeutic component 206A. In the simplest case shown here, since first vector V1 has no component along imaginary axis Im, its projection P[V1] onto real axis 222′ is simply its entire magnitude or |V1|. Thus, the first expected initial effect is the value of |V1| and is indicated by a black dot at the tip of first vector V1 on real axis 222′. Since persons skilled in the art will be familiar with the linear algebra including taking projections and calculating vector magnitudes these aspects will not be addressed herein.

Similarly, on administration, second therapeutic component 206B is represented by a second vector V2 in complex interaction space 228. The magnitude of second vector V2 is set in proportion to an initial amount of second therapeutic component 206B administered to the patient. Again, in the simplest case shown in FIG. 2, the magnitude of second vector V2 is simply set equal to first expected initial effect of second therapeutic component 206B on measurable patient response 214. Further, second vector V2 also has a projection onto one-dimensional response measure space 222, in this case a projection onto axis 222′, that is set equal to first expected initial effect of second therapeutic component 206B. In the simplest case shown here, since second vector V1 also does not have a component along imaginary axis Im, its projection P[V2] onto real axis 222′ is simply its entire magnitude or |V2|. Thus, as in the case of first vector V1, the expected initial effect is the value of |V2| and is indicated by a black dot at the tip of second vector V2 on real axis 222′.

The overall initial effect on patient response 214 of both first and second therapeutic components 206A, 206B is obtained by adding the projections P[V1], P[V2] of first and second vectors V1, V2 that represent therapeutic components 206A, 206B in complex interaction space 228. In the case shown, overall initial effect M* is equal to P[V1]+P[V2]. It is noted that overall initial effect M* can thus be negative, positive or zero.

As time elapses and new measurable patient responses 214 are collected with corresponding measurement devices or monitors, e.g., as shown in system 100 of FIG. 1, adjustments are made to a complex phase θ1 of first vector V1 to match newly collected patient responses 214.

Adjustments of complex phase θ1 cause first vector V1 to be rotated in complex interaction space 228 to yield adjusted first vector V1′. More precisely, complex phase θ1 is adjusted in accordance with expected change of the effects of first therapeutic component 226A on measurable patient response 214 with time when considered independently of any other therapeutic components. It is now the projection P[V1′] of adjusted first vector V1′ that represents the new measurable patient response 214 to first therapeutic component 226A. A person skilled in the art will recognize that the updates or adjustments to complex phase θ1 that match new patient responses 214 can be collected and tracked in a time series θ1(t). This may be advantageous in the adaptive machine learning algorithm as it provides information about the time orbit of vector V1. Additionally, the magnitude of vector V1 may be reduced in time as first therapeutic component 226A is processed and metabolized by the patient.

Similarly, adjustments are made to a complex phase θ2 of second vector V2 to match newly collected patient responses 214. As before, adjustments of complex phase θ2 cause second vector V2 to be rotated in complex interaction space 228 to yield adjusted second vector V2′. As in the case of complex phase θ1, complex phase θ2 is adjusted in accordance with expected change of the effects of second therapeutic component 226B on measurable patient response 214 with time when considered independently of any other therapeutic components. Now, the projection P[V2′] of adjusted second vector V2′ represents the new measurable patient response 214 to second therapeutic component 226B. The updates or adjustments to complex phase θ2 that match new patient responses 214 can also collected and tracked in a time series θ2(t) for second therapeutic component 226B and used in adaptive machine learning algorithm as it provides information about the time orbit of vector V2. Additionally, the magnitude of second vector V2 may be reduced in time as second therapeutic component 226B is processed and metabolized by the patient.

The overall new effect on patient response 214 of both first and second therapeutic components 206A, 206B is obtained by adding the projections P[V1′], P[V2′] of first and second vectors V1′, V2′ with adjusted complex phases θ1, θ2. In the case shown, overall new effect M*′ is equal to P[V1′]+P[V2′]. Once again, the overall new effect M*′ can thus be negative, positive or zero.

In general, more than one complex phase adjustment will match the new patient responses 214. In the present example, combinations of phase responses by −θ1 and by −θ2 would produce the same overall new effect M*′. However, all possible complex phase adjustments, i.e., θ1 as well as −θ1 to first vector V1 are kept rather than being discarded or “collapsed”.

Due to interactions, however, overall new effect M*′ will typically differ from the one computed based on varying complex phases θ1, θ2 by considering the effects of therapeutic components 206A, 206B in isolation. It is this difference that is due to interactions and it has to be accounted for by permitting additional changes to complex phases θ1, θ2. The machine learning algorithm thus has the task to learn what additional range of adjustments, in addition to those made when therapeutic components 206A, 206B are considered independently have to be made to complex phases θ1, θ2 would match the actually observed overall new effect M*′. In this step it is important to prevent a dimensional reduction in the representation that would reduce the ability to track correlations.

Conveniently, a temporal history of the range of possible phase adjustments θ1(t), θ2(t) that match patient responses 214 over time is thus compiled. It is this collection of phase adjustments that is used by the adaptive machine learning algorithm as the data set from which to learn the combined effects on patient response 214 of both first and second therapeutic components 206A, 206B.

Returning now to FIG. 1, adaptive learning module 124 provides its input to therapeutic components adjustment module 130 based on the adjustments to complex phases of the corresponding vectors that represent each one of therapeutic components 106A-E. The same process is repeated for all measurable patient responses 118A-E and for those of the therapeutic components 106A-E that affect them.

Module 130 may follow an optimization function or any other imperative, which may be as simple as to attempt to keep one or more measurable patient responses 118A-E within a prescribed range of values. Finally, module 130 is connected to therapeutic compounding module 108, which implements the adjustments to the amounts of therapeutic components 106A-E when preparing new therapeutic compound formulation 102.

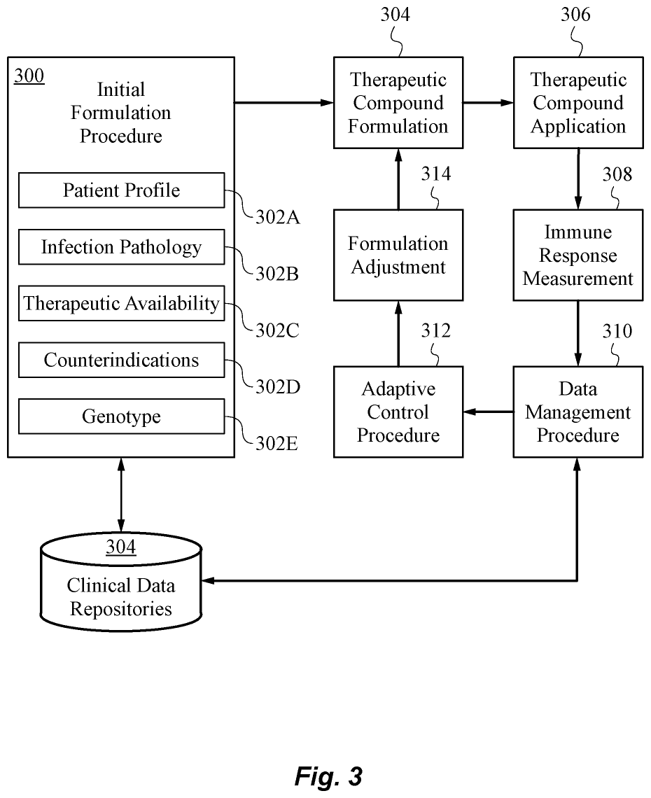

FIG. 3 illustrates a method of adjusting a therapeutic compound formulation such as formulation 102 that can be implemented in system 100 or in other systems. In an initial formulation procedure 300 a number of specific data 302 related to patient 104 are used to determine an initial formulation and determine expected initial effects on measurable patient responses. In particular, data 302 includes data about patient profile 302A, data about the patient's infection pathology 302B, data about therapeutic availability 302C, data about specific patient counterindications 302D, and data about the patient's genotype 302E. Data 302 is drawn from clinical data repositories 304. Any updates to data 302 that are determined during initial formulation procedure 300 or else during other interactions with patient 104 (e.g., follow-up with doctor or clinician) are uploaded back to clinical data repositories 304 to ensure that data 302 about patient 104 contained therein is fresh.

Initial formulation procedure 300 is followed by therapeutic compound formulation step 304, in which an actual compound formulation is prepared, e.g., formulation 102. The ready to use formulation is applied to patient 104 in therapeutic compound application step 306. Step 306 may deploy any suitable delivery mechanisms for administering formulation 102, depending on its composition. In some cases, oral administration is possible. In other cases, intra-venous, direct injection, transcutaneous delivery and still other mechanisms can be used in application step 306.

Next, immune response measurement 308 step is performed on patient 104 with whatever measurement devices are required to obtain measurable patient responses. The patient responses are selected to be macro-parameters of the type that yield reproducible and stable values rather than subjective measures or criteria. The exact choice of measurable patient responses will depend on what disease, condition, immune response, infection or malady is being addressed. In many of the cases measurable patient responses will include vital signs such as pulse rate, blood pressure and oxygenation.

The measurable patient responses obtained in step 308 are forwarded to a data management procedure 310. Here, the patient responses are conditioned and prepared. Suitable conditioning and preparation steps include filtering, noise-removal, averaging, normalization and other well-known steps to derive data in a form suitable for analysis and machine learning. In addition, properly conditioned patient responses are compared for consistency with clinical data repositories 304. When patient responses indicate that specific patient data 302 taken as the basis in the initial formulation procedure 300 deviate from expected values, then clinical data repositories 304 are updated with patient responses freshly procured in data management procedure 310. This ensures an effective closed-loop updating mechanism.

A copy of properly conditioned patient responses are sent from data management procedure 310 to adaptive control procedure 312. The latter ensures that proper representation of therapeutic compounds as vectors in complex interaction space and patient responses as one-dimensional response measure spaces is applied to the data. Further, adaptive control procedure 312 deploys an adaptive machine learning approach to derive adjustments to the complex phases of the vectors to reflect data coming in from successive measurements of patient responses. In accordance with the invention, all candidate complex phase adjustments are kept and no dimensional reduction in the representation is performed by the adaptive machine learning approach.

The output of adaptive control procedure 312 includes data on how therapeutic components of the formulation should be adjusted to achieve desired patient responses. This output is provided to formulation adjustment step 314 in which relative values or amounts of therapeutic components are calculated and processed in a manner that is deployed in therapeutic compound formulation step 304. This connection completes another closed-loop feedback that allows for personalized adjustments to therapeutic compound formulation 102 that is administered to patient 104 in accordance with the method.

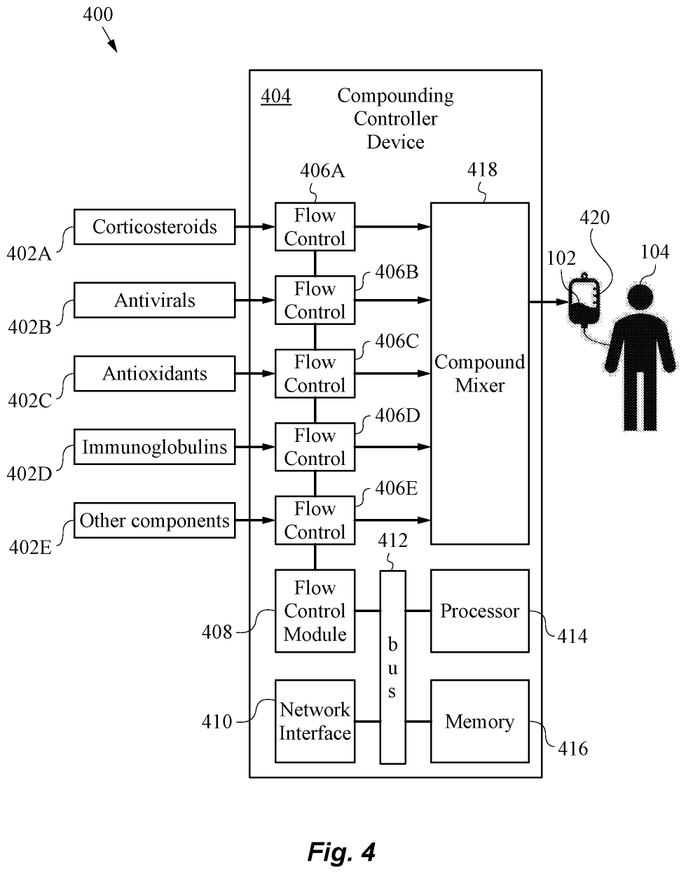

FIG. 4 shows a diagram of a compounding controller 400 that is used in preparing initial therapeutic compound formulation 102 and adjusted therapeutic compound formulation 102′. Compounding controller 400 can be used in steps 300, 304 and 314 of the method described above and shown in FIG. 3 or in another embodiment of the method of invention where the therapeutic compound formulation admits of mixing of the therapeutic components. Also, compounding controller 400 can be integrated into system 100 illustrated in FIG. 1 and described above.

Compounding controller 400 receives as inputs therapeutic components 402A-E. In this example therapeutic component 402A is a corticosteroid, component 402B is an antiviral, component 402C is an antioxidant, component 402D is an immunoglobulin and component 402E is still another agent that can be mixed into the compound. Each therapeutic component 402A-E is received in a compounding controller device 404 in a quantity that is regulated by corresponding intake or flow controls 406A-E. In turn, flow controls 406A-E are driven by a flow control module 408 that is informed about the relative amounts of therapeutic components 402A-E to be admitted into compounding controller device 404 according to initial formulation procedure of step 300 or formulation adjustment of step 314 described in FIG. 3.

In the present embodiment, compound controller device 404 has a network interface 410 connected to flow control module 408 via an internal bus 412. A processor 414 and a memory 416 are also present on-board device 404 and they are connected to bus 412. Thus, information about initial formulation procedure of step 300 or formulation adjustment of step 314 are received by compounding controller device 404 via network interface 410 and provided to flow control module 408 with any requisite processing and data recall functions performed by processor 414 and memory 416, as appropriate. For example, processor 414 and memory 416 may keep a log of formulation procedures and adjustments previously made and any new initial formulation or adjusted formulation may be corroborated or cross-checked against such log. Additionally, memory 416 may contain historic information that processor 414 may use in validating any updates sent to flow control module 408. Such functions and other associated flow control issues will be understood by those skilled in the art and are thus not addressed herein.

Compound controller device 404 has a compound mixer 418 into which flow controls 406A-E input their corresponding therapeutic components 402A-E. Mixer 418 is selected based on the material properties of therapeutic components 402A-E as well as the expected range of material properties that will be exhibited by final therapeutic compound formulation 102 or adjusted therapeutic compound formulation 102′. Relevant material properties include miscibility, relative viscosities, stability, sedimentation and other relevant material properties.

Compound mixer is connected to administration mechanism 420 that delivers therapeutic compound formulation 102 (or adjusted formulation 102′) to patient 104. Although mechanism 420 is an intra-venous delivery device in the present case, it should be noted that other mechanisms, depending on the final state of formulation 102 may be used depending on the type of therapeutic formulation 102.

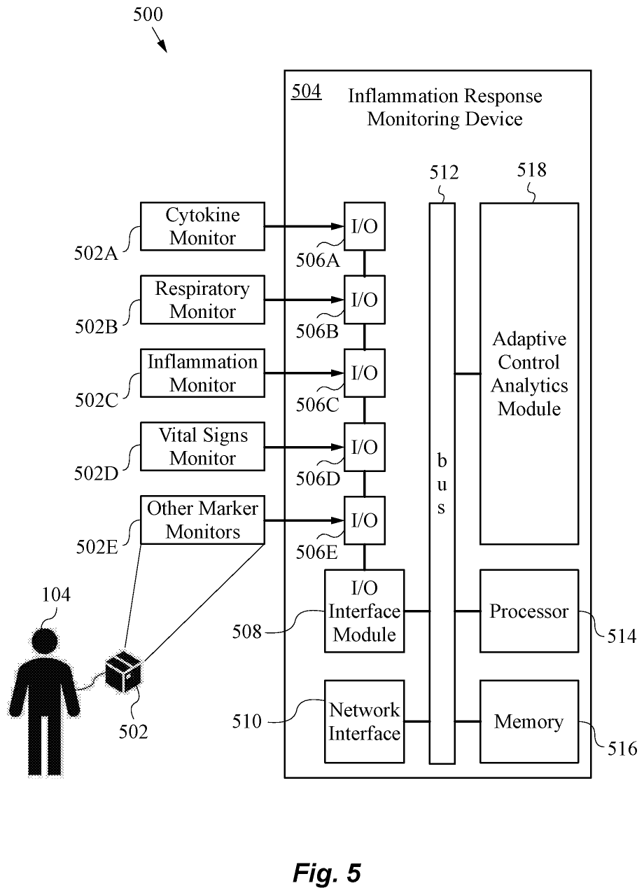

FIG. 5 shows an inflammatory response monitor 500 that may be integrated into system 100 of FIG. 1. In others words, immune response monitor 500 may be integrated into a single device that embodies an integrated version of measurement devices and monitors 112 of FIG. 1. Also, inflammatory response monitor 500 can be used in practicing steps 308, 312 and 314 of the method illustrated in FIG. 3 and described above, or in still other methods according to the invention.

In the present example, response monitor 500 has an integrated monitoring device 502 that has a cytokine monitor 502A, a respiratory monitor 502B, an inflammation monitor 502C, a vital signs monitor 502D and still other marker monitors 502E for obtaining selected measurable patient responses. Monitors 502A-E feed their measured patient responses into an inflammation response monitoring device 504. More precisely, measured patient responses from each one of monitors 502A-E are received via a dedicated input/output or I/O port 506A-E individually assigned to corresponding monitors 502A-E. In this manner the various data types along with their signal characteristics can be processed separately and in different ways, as appropriate for each signal type. A person skilled in the art will appreciate that this layout is very flexibly as it permits the user to perform independent signal processing and conditioning (e.g., thresholding, filtering, averaging, etc.) on each set of measured patient responses.

In turn, I/O ports 506A-E deliver their signals to an input/output (I/O) interface module 508 that is informed about any independent pre-processing of signals representing measured patient responses. In fact, I/O interface module 508 preferably is in charge of correcting and/or controlling the activity of each I/O port 506A-E.

In the present embodiment, inflammation response monitoring device 504 has a network interface 510 connected to I/O interface module 508 via an internal bus 512. A processor 514 and a memory 516 are also present on-board device 504, and they are also connected to bus 512. Thus, information representing measured patient responses can be combined and processed into the form required in immune response measurement step 308 of the method described in conjunction with FIG. 3. Any requisite processing and data recall functions to place the immune response data can be performed by processor 514 and memory 516 as appropriate. For example, processor 514 and memory 516 may keep a log of processing applied to the data representing measured patient responses and adjustments previously made such that the data can be corroborated or cross-checked against such log. Additionally, memory 516 may contain historic information that processor 514 may use in validating any data. Such functions and other associated signal processing issues will be understood by those skilled in the art and are thus not addressed herein. These actions typically fall within the step of data management procedure 310 of FIG. 3.

Inflammation response monitoring device 504 has an adaptive control analytics module 418 into which all conditioned and processed data containing measured patient responses is sent. Adaptive control analytics module 518 can be in charge of implementing adaptive control procedure 312 of the method shown in FIG. 3. In particular, the adaptive machine learning according to the invention can be performed by analytics module 518—indeed, module 518 can even be connected to external resources for this reason.

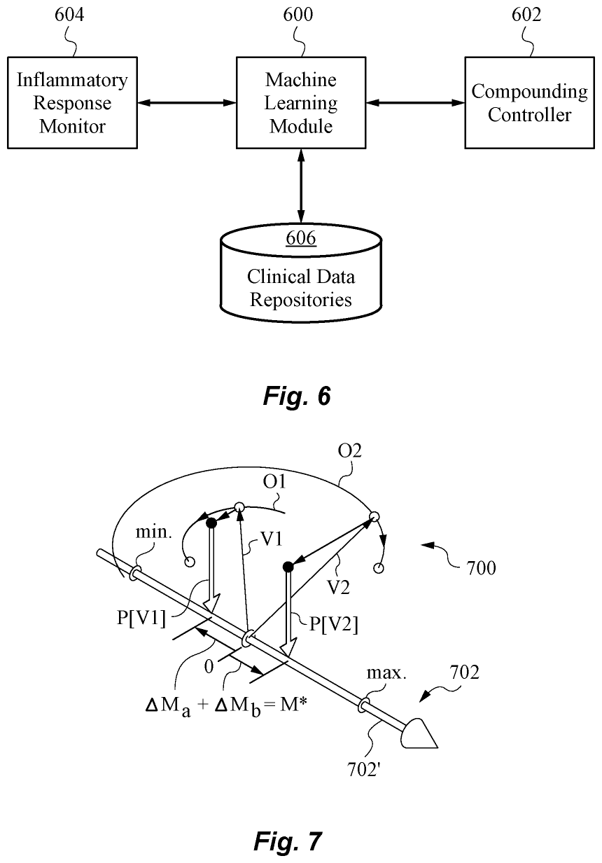

FIG. 6 is a general diagram illustrating the overall approach to adaptive control and adaptive machine learning in methods and systems according to the invention. A machine learning module 600 is at the center of the approach. Module 600 is in communication with a compounding controller 602. Specifically, module 600 receives inputs from and provides outputs, such as adjustments based on its learning to a compounding controller 602.

Module 600 is also in communication with an inflammatory response monitor 604. Module 600 receives inputs used for learning from inflammation response monitor 604. Also, module 600 provides outputs to inflammation response monitor 604 to adjust its operation.

Finally, machine learning module 600 is also in communication with clinical data repositories 606. Data about the patient is drawn from data repositories to set up the initial conditions for machine learning. More specifically, the data from repositories 606 is used in making the selection of therapeutic components as well as measurable patient indications along with expected initial effects. As learning module 600 learns adjustments, especially personalized adjustments for a particular patient, it provides its learned adjustments back to data repositories 606. Thus, the approach of the invention can be applied for making future personalized adjustments of the therapeutic components of the compound formulation intended for the same patient based on their own measurable patient responses. However, data thus obtained through learning can also be used for a type or group of patients to whom the learning gathered from any particular patient can also apply.

FIG. 7 is a diagram illustrating another exemplary representation of two therapeutic components and a response measure space in a higher-dimensional complex interaction space 700. The therapeutic components of a compound formulation are represented in this still higher-dimensional complex space 700. A particular measurable patient response is mapped to or represented in a response measure space 702 that is assigned to a real and one-dimensional space spanned by axis 702′. The origin of axis 702′ is placed at the value of the measurable patient response prior to the administration of the two therapeutic components contained in the compound formulation.

Upon administration, the first therapeutic component is represented by a first vector V1 in complex interaction space 700. The magnitude |V1| of first vector V1 is set in proportion to an initial amount of first therapeutic component administered to the patient. In the simplest case, the magnitude |V1| is simply set equal to first expected initial effect of the first therapeutic component on the measurable patient response. Further, first vector V1 also has a projection onto one-dimensional response measure space 702, in this case a projection onto axis 702′, that is set equal to first expected initial effect of the first therapeutic component. In the simplest case shown here, a projection P[V1] of first vector V1 onto real axis 702′ is shown with the aid of two orthogonal arrows.

Similarly, on administration, the second therapeutic component is represented by a second vector V2 in complex interaction space 700. The magnitude |V2| of second vector V2 is set in proportion to an initial amount of the second therapeutic component administered to the patient. Again, the magnitude of second vector V2 is simply set equal to first expected initial effect of the second therapeutic component on the measurable patient response. Further, second vector V2 also has a projection P[V2] onto one-dimensional response measure space 702, in this case a projection onto axis 702′ shown with the aid of two orthogonal arrows.

Based on prior data and knowledge, in the absence of interactions, vectors V1 and V2 are taken to evolve in time along respective orbits O1 and O2 in space 700 so as to correspond to independent measurable effects expected over time due to administration of the two therapeutic components. Differently stated, orbits O1 and O2 are taken here to represent the time evolution of vectors V1 and V2, rather than indicating them by angles as in the previous embodiment of FIG. 2. More precisely, when the two therapeutic components represented by vectors V1, V2 are considered independently, then vectors V1, V2 are expected to evolve or travel in time along orbits O1, O2 as indicated by the arrows indicated along orbits O1, O2.

The overall initial effect on the patient response of both the first and second therapeutic components is obtained by adding the projections P[V1], P[V2] of first and second vectors V1, V2 that represent these therapeutic components in complex interaction space 700. In the case shown, overall initial effect M* is equal to P[V1]+P[V2]. It is noted that overall initial effect M* can thus be negative, positive or zero.

As time elapses and new measurable patient responses are collected with corresponding measurement devices or monitors, e.g., as shown in system 100 of FIG. 1, adjustments are made to the location of first vector V1 along orbit O1 to match newly collected patient responses. Adjustments to the location of the tip of first vector V1 along orbit O1 in complex interaction space 700 are shown with the aid of a ball on orbit O1. The location of this ball is adjusted in accordance with expected change of the effects of the first therapeutic component on the measurable patient response with time when considered independently of the second therapeutic component. Similarly, adjustments are made to the location of second vector V2 along orbit O2 to match newly collected patient responses. Adjustments to the location of the tip of second vector V2 along orbit O2 are also visualized with the aid of a ball on orbit O2. The location of this ball is adjusted in accordance with expected change of the effects of the second therapeutic component on the measurable patient response with time when considered independently of the first therapeutic component.

The overall new effect on the patient response mapped to response measure space 702 spanned by axis 702′ due to both first and second therapeutic components is obtained by adding the projections P[V1′], P[V2′] of first and second vectors V1′, V2′ at the adjusted positions along orbits O1 and O2. Again, more than one set of adjusted positions along orbits O1, O2 will match the new patient responses. All possible orbit adjustments are kept rather than being discarded or “collapsed”.

Due to interactions, overall new effect M*′ will typically differ from the one computed based on varying positions of tips of vectors V1′, V2′ along orbits O1, O2 that are obtained by considering the effects of the two therapeutic components in isolation. It is this difference that is due to interactions and it has to be accounted for by permitting additional changes to positions of tips of vectors V1′, V2′ along orbits O1, O2. The machine learning algorithm thus has the task to learn what additional range of adjustments, in addition to those made when the therapeutic components are considered independently have to be made to match the actually observed overall new effect M*′. In this step it is important to prevent a dimensional reduction in the representation that would reduce the ability to track correlations.

Clearly, because of the large space of possible adjustments along orbits O1, O2 for even just two therapeutic components it is important to prevent an explosion in the total correlations that can be tracked by keeping to the total number of therapeutic components relatively small. For example, the total number of these additional therapeutic components is kept at just a few, such as 5 to 10 and preferably at less than 20. Additionally, all measurable patient response should be monitored or measured at a higher than normal rate or frequency in order to provide sufficient data to the adaptive learning algorithm.

In some embodiments a series of measurements of the selected patient response and any prior knowledge are used to set constraints on the behavior/dynamics of the complex vectors in the complex interaction space. In those cases, the knowledge is used to place additional bounds or constraints on the orbits or phase angles of the vectors representing the therapeutic components.

The method of the invention does not attempt to construct a model of cell biology or signaling pathways with any mechanistic details. The method is also not attempting to predict cellular systems. The method concentrates instead on the behavior of a single macro-parameter at a time by taking its action in isolation at first, and then observing what takes place when there are interactions. The parameter being measured in the patient should be chosen such that it reacts to each component in a compound that contains relatively small numbers of components in order to contain the combinatorial explosion in the complex space. We target a solution space of N preferably up to 5 and at most 10 inputs or components/compounds at a time.

It will be noted that the approach taught herein is designed to track are histories or evolution in time of measurements of the macro-parameter. From the best fits the dynamics are postulated as candidates in the high-dimensional complex space. This type of approach is also sometimes referred to as de novo by those skilled in the art. It will be noted that many possible interaction dynamics will initially correspond/match the observed/measured results of the chosen parameter. Reduction is not performed unless a clear indication is found for ruling out of a candidate interaction.

It will be evident to a person skilled in the art that the present invention admits of various other embodiments. Therefore, its scope should be judged by the claims and their legal equivalents.

1. A method for personalized adjustment of therapeutic components in a therapeutic compound formulation administered to a patient based on a measurable patient response, said method comprising:
a) assigning said measurable patient response to a one-dimensional response measure space;
b) administering a first therapeutic component having a first expected initial effect on said measurable patient response based on a clinical data repository;
c) representing said first therapeutic component in a complex interaction space by a first vector having:
i) a magnitude proportional to an initial amount of said first therapeutic component;
ii) a projection onto said one-dimensional response measure space equal to said first expected initial effect;
d) determining adjustments to a complex phase of said first vector to match said measurable patient response;
e) administering a second therapeutic component having a second expected initial effect on said measurable patient response based on said clinical data repository;
f) representing said second therapeutic component in said complex interaction space by a second vector;
wherein correlations between said first therapeutic component and said second therapeutic component of said therapeutic compound formulation are tracked in said complex interaction space without applying dimensional reduction.

2. The method of claim 1, with said second vector having:
a) a magnitude proportional to an initial amount of said second therapeutic component;
b) a projection onto said one-dimensional response measure space equal to said second expected initial effect; and
and further determining adjustments in said complex phase of said first vector and in a complex phase of said second vector to match said measurable patient response.

3. The method of claim 2, further comprising an adaptive learning algorithm for performing said step of determining adjustments in said complex phase of said first vector and of said second vector.

4. The method of claim 3, wherein said adaptive learning algorithm retains said first vector and said second vector representation in said complex interaction space without applying dimensional reduction and without setting causal connections.

5. The method of claim 3, wherein said adaptive learning algorithm produces a history of measurements of said measurable patient response and said adjustment in said complex phase of said first vector and of said second vector is based on said history.

6. The method of claim 2, wherein said one-dimensional response measure space is a real space.

7. The method of claim 1, wherein said first therapeutic component and said second therapeutic component are chosen in an initial formulation procedure based on at least one datum selected from the group consisting of a patient profile, an infection pathology, a therapeutic availability, a counterindication, a genotype.

8. The method of claim 1, wherein said measurable patient response is selected from the group consisting of cytokines, a respiratory function, an inflammation condition, a vital sign.

9. The method of claim 1, wherein said first therapeutic component and said second therapeutic component are selected from the group consisting of corticosteroids, antivirals, antioxidants, immunoglobulins.

10. The method of claim 1, wherein said therapeutic compound formulation is prepared by an immunomodulator therapeutic compounding module.

11. The method of claim 1, further comprising applying additional therapeutic components, whereby the total number of said additional therapeutic components is less than 20.

12. The method of claim 1, wherein said measurable patient response is measured with a higher than normal frequency.

13. A computer-implemented adaptive learning method for personalized adjustments of therapeutic components in a therapeutic compound formulation administered to a patient based on measurable patient responses, said method comprising:
a) assigning a one-dimensional response measure space to said measurable patient responses;
b) setting a first expected initial effect on said measurable patient responses to the administration of a first therapeutic component based on a clinical data repository;
c) representing said first therapeutic component in a complex interaction space by a first vector having:
i) a magnitude proportional to an initial amount of said first therapeutic component;
ii) a projection onto said one-dimensional response measure space equal to said first expected initial effect;
d) learning adjustments to a complex phase of said first vector to match said measurable patient responses;
e) setting a second expected initial effect on said measurable patient responses to the administration of a second therapeutic component based on said clinical data repository;
f) representing said second therapeutic component in said complex interaction space by a second vector;
wherein correlations between said first therapeutic component and said second therapeutic component of said therapeutic compound formulation are learned in said complex interaction space without dimensional reduction.

14. The computer-implemented adaptive learning method of claim 13, with said second vector having:
a) a magnitude proportional to an initial amount of said second therapeutic component;
b) a projection onto said one-dimensional response measure space equal to said second expected initial effect; and
and further learning adjustments in said complex phase of said first vector and in a complex phase of said second vector to match said measurable patient responses.

15. The computer-implemented adaptive learning method of claim 14, further comprising retaining said first vector and said second vector representation in said complex interaction space without setting causal connections.

16. The computer-implemented adaptive learning method of claim 14, further comprising keeping a history of measurements of said measurable patient responses, and wherein said learned adjustments in said complex phase of said first vector and of said second vector are based on said history.

17. The computer-implemented adaptive learning method of claim 13, wherein said first therapeutic component and said second therapeutic component are chosen in an initial formulation procedure based on at least one datum selected from the group consisting of a patient profile, an infection pathology, a therapeutic availability, a counterindication, a genotype.

18. The computer-implemented adaptive learning method of claim 13, wherein said measurable patient responses are selected from the group consisting of cytokines, a respiratory function, an inflammation condition, a vital sign.

19. The computer-implemented adaptive learning method of claim 13, wherein said first therapeutic component and said second therapeutic component are selected from the group consisting of corticosteroids, antivirals, antioxidants, immunoglobulins.

20. The computer-implemented adaptive learning method of claim 13, wherein said therapeutic compound formulation is prepared by an immunomodulator therapeutic compounding module.

21. The computer-implemented adaptive learning method of claim 13, further comprising administration of additional therapeutic components, whereby the total number of said additional therapeutic components is less than 20.
